(19) United States
(12) Patent Application Publication
(10) Pub. No.: US 2009/0129648 A1
(43) Pub. Date: May 21, 2009

(54) Method of reducing imaging time in propeller-MRI by under-sampling and iterative image reconstruction

(76) Inventors: Konstantinos Arfanakis, Chicago, IL (US); Mark A. Anastasio, Aurora, IL (US); Ashish A. Tamhane, Chicago, IL (US)

Correspondence Address: Mark D. Swanson;Pauley Petersen & Erickson, Suite 365, 2800 West Higgins Road, Hoffman Estates, IL, 60169, US

(21) Appl. No.: 11/985,544

(22) Filed: Nov. 15, 2007

(51) Int. Cl. A61B 5/055

(52) U.S. Cl. 382/131

(57) ABSTRACT

A method for reducing PROPELLER MRI data acquisition times, by combining k-space under-sampling and iterative reconstruction using NUFFT, while maintaining similar image quality as in PROPELLER MRI with sufficient k-space sampling. Iterative image reconstruction using NUFFT minimizes image artifacts produced with conventional PROPELLER image reconstruction in under-sampled acquisitions. The data acquisition and image reconstruction parameters are selected in order to achieve image quality similar to that of sufficiently-sampled PROPELLER acquisitions for significantly shorter imaging time. An advantage of using under-sampled PROPELLER imaging is a reduction in acquisition time by as much as 50% without introducing significant artifacts, and while maintaining other benefits of PROPELLER imaging.

### BACKGROUND OF THE INVENTION

This invention relates generally to magnetic resonance imaging and, more particularly, to PROPELLER magnetic resonance imaging.

Magnetic resonance imaging (MRI) is a known technique for obtaining images of the inside of an object under investigation, such as a patient. An MRI apparatus generates a static magnetic field around at least a portion of the object, so as to order or align the random ordered nuclear spins of the nuclei in the object. A radio-frequency (RF) antenna system is also a part of the apparatus, and includes an RF transmission coil and at least one RF reception coil. In some instances, the RF transmission coil and the RF reception coil may be the same. RF energy is irradiated into the examination subject by the RF transmission coil, causing magnetic resonance signals to be generated in the subject, which are detected (received) by the RF reception coil or coils.

The received, analog magnetic resonance signals are converted into digital signals, and represent a so-called raw data set. The raw data set is obtained in the Fourier domain, also known as k-space. By means of an inverse Fourier transformation, the data in k-space are transformed into image data.

When MRI is used with a live subject, the subject is required to remain generally still during data acquisition. As it is often difficult to obtain complete stillness, efforts have been made to create MRI methods that are less affected by motion and/or to reduce the generally long imaging time for obtaining the data sets.

One known MRI imaging technique, called echo-planar imaging (EPI), separates a train of readout gradients by small phase encoding gradients and acquires the complete k-space image within one excitation without a 180° refocusing pulse. Another technique is referred to as spin-echo sequence (SE), where each line of k-space is acquired after one excitation and a 180° refocusing pulse. However, if a train of 180° refocusing pulses is included in SE, and multiple k-space lines are acquired for each excitation, then the sequence is referred to as fast spin echo (FSE). Even though imaging time for EPI acquisitions is smaller compared with FSE, images obtained with EPI are severely affected by magnetic field inhomogeneity-related artifacts. In contrast, FSE is immune to these artifacts, but FSE has a longer imaging time that causes severe motion related artifacts, particularly in the case of uncooperative patients. These shortcomings of conventional sequences are answered to a degree by a MRI technique called PROPELLER (Periodically Rotated Overlapping Parallel Lines with Enhanced Reconstruction).

PROPELLER MRI is a non-Cartesian data acquisition technique that is rapidly attracting attention due to its typically greatly reduced sensitivity to various sources of image artifacts. PROPELLER data acquisitions follow a multi-shot FSE approach, in which several k-space lines are acquired after each excitation, forming a blade that is then rotated around its center and acquisition is repeated to cover k-space, as shown in FIG. 1. Since PROPELLER MRI is based on FSE techniques, the images produced contain significantly fewer magnetic field inhomogeneity-related artifacts than EPI, and do not suffer by warping due to eddy currents. Also, a central disk of k-space is acquired in each blade that can be used as a 2D navigator to correct data between shots without requiring additional echoes. PROPELLER acquisitions are radial in nature and thus uncorrected errors are expressed in a benign fashion, similar to projection reconstruction methods.

However, the imaging time in PROPELLER MRI is considerably longer than in EPI, particularly since PROPELLER is based on multi-shot FSE. Furthermore, the imaging time in PROPELLER is generally at least 50% longer than in conventional multi-shot FSE, due to the over-sampling that occurs in the central region of k-space when using the PROPELLER sampling grid. In the most recent form of PROPELLER imaging, named TURBOPROP, data acquisition is accelerated by reading out multiple lines of k-space after each 180° pulse, similar to the gradient and spin echo (GRASE) sequence, thereby increasing the number of lines per blade, and reducing the total number of blades required to cover k-space. In addition to the shorter imaging time, the increased number of lines per blade in TURBOPROP leads to more robust motion correction. However, even in TURBOPROP-MRI, multiple excitations are required for each image, and thus the acquisition time is still longer than that of EPI. Further acceleration can be achieved with a technique referred to as PROPELLER EPI, which does not contain 180° pulses, and each blade is acquired with an EPI acquisition window following an excitation pulse. However, PROPELLER EPI images are typically contaminated by susceptibility-related artifacts and blurring, similar to conventional EPI. Also, multiple excitations are required for each image, and thus the acquisition time is still longer than that of EPI.

There is a need for an improved MRI technique that is faster and/or less susceptible to image artifacts.

### SUMMARY OF THE INVENTION

A general object of the invention is to provide an improved MRI imaging technique. More particularly, an object of the invention is to provide a method of reducing the number of MR scans and k-space data sets required for obtaining an MR image, without artifacts.

A more specific objective of the invention is to overcome one or more of the problems described above.

The general object of the invention can be attained, at least in part, through a method of obtaining a magnetic resonance (MR) image. The method includes conducting a plurality of MR scans, acquiring a plurality of k-space data sets from the plurality of MR scans, transforming the plurality of k-space data sets to an image space using an iterative reconstruction process, and displaying the magnetic resonance image.

The invention further comprehends a method of obtaining a MR image including conducting a plurality of PROPELLER MR scans, acquiring a plurality of k-space data sets from the plurality of MR scans, transforming the plurality of k-space data sets to an image space using non-uniform fast Fourier transform, and displaying the magnetic resonance image.

In one embodiment of the invention, the transition between k-space and image space is performed with a non-uniform fast Fourier transform (NUFFT) operator and its adjoint operator. A quadratic penalty weighted least squares function is used in order to minimize the total energy in the image. The data acquisition and image reconstruction parameters are selected in order to achieve image quality similar to that of fully-sampled PROPELLER acquisitions for significantly shorter imaging time.

Other objects and advantages will be apparent to those skilled in the art from the following detailed description taken in conjunction with the appended claims and drawings.

### BRIEF DESCRIPTION OF THE DRAWINGS

FIG. 1 generally illustrates the formation of a PROPELLER k-space sampling pattern for explanatory purposes.

FIG. 2 is a representation of a PROPELLER k-space sampling pattern.

FIG. 3 illustrates PROPELLER sampling patterns with (A) 12 blades, (B) 10 blades, (C) 8 blades, and (D) 6 blades, and objects imaged with each sampling pattern. All sampling patterns have 16 lines per blade and 128 sample points per line. Sampling pattern (A) represents full-sampling, and sampling patterns (B), (C), and (D) represents different levels of under-sampling.

### DETAILED DESCRIPTION OF THE INVENTION

The present invention provides an iterative image reconstruction technique that reduces image artifacts in under-sampled PROPELLER acquisitions. The invention also includes software that allows the method to be easily implemented in existing MRI machines. The advantage of using under-sampled PROPELLER imaging with the image reconstruction method of this invention is a reduction in acquisition time, such as by as much as 50%, without introducing significant artifacts and while maintaining other benefits of PROPELLER imaging.

While the invention is discussed herein with particular reference to PROPELLER MRI, the invention is not intended to be so limited. For example, the method described here allows reconstruction of images without significant artifacts for a fraction of the imaging time required for full sampling of k-space in PROPELLER, TURBOPROP, or PROPELLER-EPI. Thus, this invention is not only applicable to the PROPELLER sequence but the PROPELLER family of sequences including, without limitation, TURBOPROP, and PROPELLER-EPI.

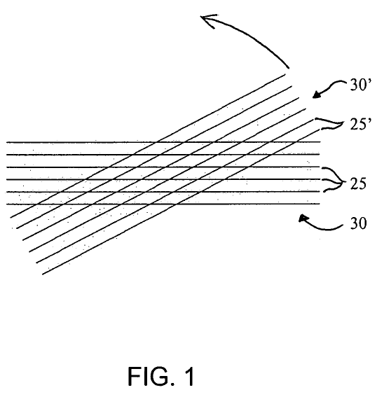

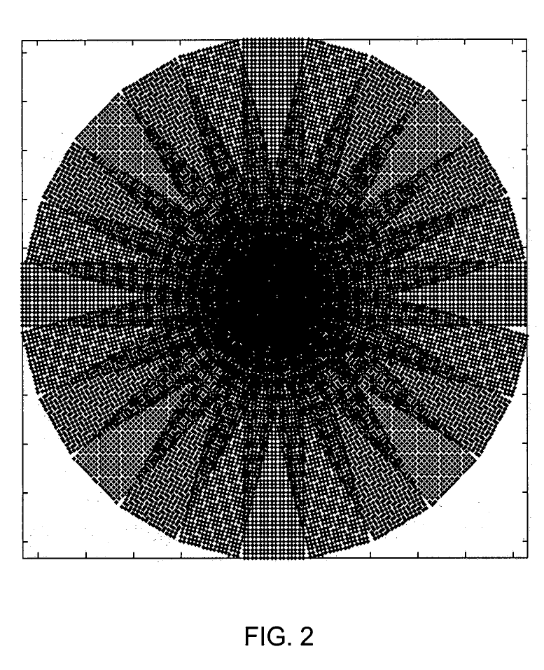

In one embodiment of this invention, there is provided a method of obtaining a magnetic resonance (MR) image. The method includes conducting a plurality of MR scans, such as with a known and available MRI apparatus, and acquiring a plurality of k-space data sets from the plurality of MR scans. The method of this invention is particularly useful in conjunction with the PROPELLER MRI technique. As mentioned above and shown in FIG. 1, in PROPELLER MR scans several k-space lines 25 (such as 16 lines, as shown in FIG. 2) are acquired after each excitation, forming a blade 30 that is then rotated around its center and acquisition is repeated (lines 25′ and blade 30′) to cover k-space. FIG. 2 illustrates an exemplary PROPELLER k-space sampling pattern having 12 blades with 16 lines per blade. The plurality of MR scans for use in one embodiment of this invention thus includes a plurality of PROPELLER blade MR scans, and the plurality of k-space data sets includes a plurality of k-space lines.

As mentioned above, the method of this invention achieves an image quality similar to that of fully-sampled PROPELLER acquisitions using under-sampling of k-space, thereby allowing for a significantly shorter imaging time. In MRI, Cartesian k-space sampling schemes that satisfy the following relationship are called sufficiently sampled:

Δk=1/FOV (1)

where Δk is the maximum distance between adjacent samples in k-space and FOV is the field of view in image space. Sampling schemes with Δk>1/FOV are called under-sampled, and are used to reduce the number of samples and accelerate the imaging process. However, images reconstructed from under-sampled acquisitions generally suffer from image artifacts caused by aliasing. The amount of artifacts depends on the degree of under-sampling. In non-Cartesian sampling schemes, there exist similar sampling criteria that define full sampling and under-sampling, and similar artifacts appear in images reconstructed from under-sampled acquisitions.

In PROPELLER-based sequences, data are sampled non-uniformly across k-space. As will be evident from FIGS. 1 and 2, the sampling density is higher near the center of k-space than towards the edges. Thus, in PROPELLER-based sequences, a sampling pattern that provides full sampling satisfies an equation similar to Equation (1) only at the periphery of k-space, while the central region of k-space is over-sampled. More specifically, if B is the number of blades of a PROPELLER sampling pattern, L is the number of lines per blade, and N is the number of samples per line, then if: 2*L*B=π*N, the criteria mentioned in Equation (1) are maintained at the peripheral k-space is called sufficiently sampled. It is common practice to acquire 128 points per line (N) in PROPELLER, while keeping the distance between adjacent points=1/FOV. Furthermore, high quality data can be obtained in PROPELLER when the maximum number of lines in each blade is approximately 16, since all lines in a blade are acquired after a single excitation, and signal decays exponentially with time following the excitation. Thus, the number of blades required to sufficiently sample k-space in PROPELLER is 12.

Under-sampling in PROPELLER-MRI can be achieved in the following three ways: (a) by increasing the distance between samples and reducing the number of samples per line; (b) by increasing the distance between lines and decreasing the number of lines per blade while keeping the number of blades constant; and (c) by increasing the distance between lines while keeping the number of lines per blade the same and reducing the number of blades. Under-sampling schemes (a) and (b) are expected to only lead to a minor reduction in imaging time, since they only reduce the time for acquisition of a single blade, and they don't reduce the number of blades, which is linearly related to the imaging time. Scheme (c) actually reduces the number of blades, and therefore the number of excitations and the total imaging time. Thus, scheme (c) is expected to lead to the most significant reduction in imaging time for PROPELLER.

If a k-space sampling pattern that provides sufficient sampling in PROPELLER contains 12 blades, 16 lines per blade, and 128 samples per line, an example of an under-sampled pattern following scheme (c) would contain, for example, 6 blades, 16 lines with spacing of 2/FOV, and 128 samples per line, for a 50% reduction in imaging time. Scheme (c) is thus desirable in one embodiment of this invention for accelerating PROPELLER-based sequences.

If conventional gridding (conventional PROPELLER image reconstruction technique) is used to reconstruct images from PROPELLER data obtained with any of the under-sampling schemes mentioned above (a-c), these images will contain significant artifacts. The nature of these artifacts is similar to that of artifacts produced in other types of under-sampled MRI acquisitions. In one embodiment of the invention, under-sampling scheme (c) is used in combination with an iterative reconstruction based on the non-uniform fast Fourier transform (NUFFT) to reconstruct images with significantly reduced artifacts.

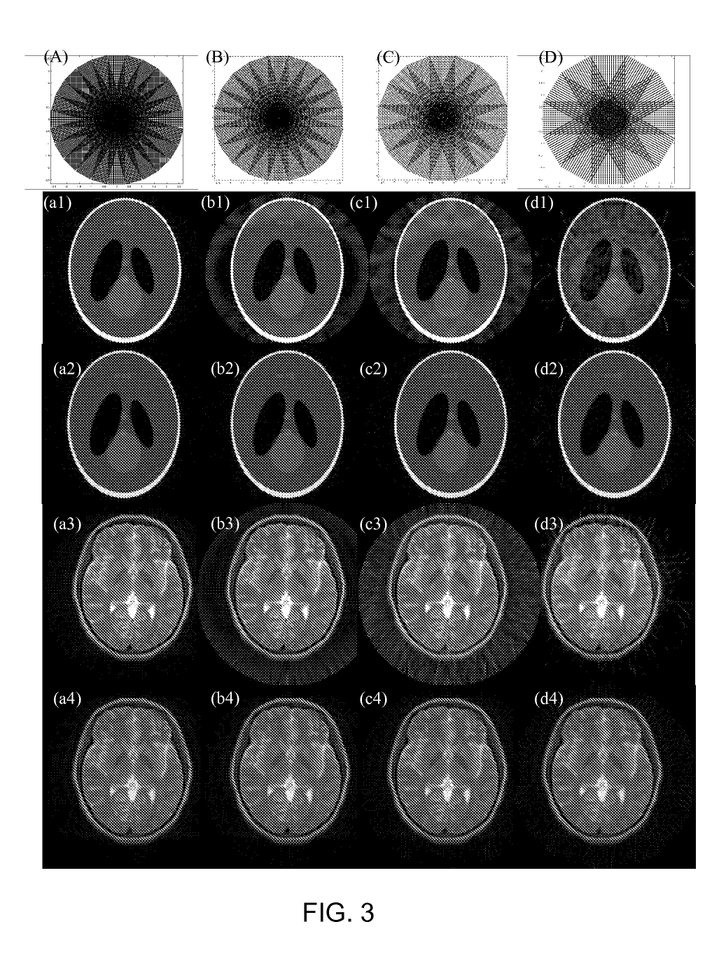

The effects of under-sampling on reconstructed images are demonstrated in FIG. 3. Under-sampling schemes were produced by increasing the distance between adjacent lines in one blade, while decreasing the total number of blades. In FIG. 3, each column represents images produced with the PROPELLER sampling pattern shown in the first row. Sampling pattern (A) includes 12 blades, sampling pattern (B) includes 10 blades, sampling pattern (C) includes 8 blades, and sampling pattern (D) includes 6 blades. All sampling patterns have 16 lines per blade and 128 points per line. The distance between adjacent lines is {1/FOV, 1.26/FOV, 1.57/FOV, 2/FOV} for {(A), (B), (C), (D)} respectively.

FIG. 3 includes images reconstructed using both conventional gridding and with an iterative reconstruction approach using NUFFT according to this invention. Images (a1), (b1), (c1), and (d1) were reconstructed from sampling patterns (A), (B), (C), and (D), respectively, using conventional MRI gridding. Images (a2), (b2), (c2), and (d2) were reconstructed according to the method of this invention by iterative reconstruction using NUFFT from sampling patterns (A), (B), (C), and (D), respectively. Similarly human brain images (a3), (b3), (c3), and (d3) were reconstructed from sampling patterns (A), (B), (C), and (D), respectively, using conventional MRI gridding, and (a4), (b4), (c4), and (d4) were reconstructed according to the method of this invention by iterative reconstruction using NUFFT from sampling patterns (A), (B), (C), and (D), respectively.

As evident in FIG. 3, as the degree of under-sampling increased, the artifacts caused due to aliasing also increased in images reconstructed using conventional gridding. However, iterative reconstruction using NUFFT according to the method of this invention produced images with significantly reduced artifacts even when under-sampling by 50% (50% reduction in imaging time).

As mentioned earlier, the raw MRI signal does not represent intensities in image-space, but instead the spatial frequency content of the imaged object. Thus, in MRI the raw signal corresponds to intensities in spatial frequency space (k-space). Under the conditions of spin-warp imaging and using the appropriate time-varying magnetic field gradients, G(τ), the signal is given by the following equation:

$$s ( t ) = \int p ( x ) {e}^{γ {\int }_{0}^{t} G ( τ ) x d τ} d x \qquad ( 2 )$$

where s is the signal at spatial frequency k, ρ(x) is the density of protons at position x in image space, γ is the gyromagnetic ratio, and k is given by:

$$k ( t ) = \frac{γ}{2 Π} {\int }_{0}^{t} G ( τ ) d τ \qquad ( 3 )$$

The function k(t) can be interpreted as the sampling trajectory in k-space. In two dimensions, the location of k-space samples k(t)=[kₓ(t), k_{y}(t)] of the imaged object is given by:

$${k}_{x} ( t ) = \frac{γ}{2 Π} {\int }_{0}^{t} {G}_{x} ( τ ) d τ , {k}_{y} ( t ) = \frac{γ}{2 Π} {\int }_{0}^{t} {G}_{y} ( τ ) d τ \qquad ( 4 )$$

The complex signal is given by:

$$s ( t , x , y ) = \int p ( x , y ) {e}^{γ {\int }_{0}^{t} ( {G}_{x} ( τ ) x + {G}_{y} ( τ ) y ) d τ} d x \qquad ( 5 )$$

In conventional two-dimensional (2D) MR imaging, a constant Gₓ gradient (readout gradient) is turned on during signal readout, which causes frequency encoding along the x-axis and allows sampling of signals located in different positions along the kₓ axis (Equation 4). In addition, in conventional 2D MR imaging a G_{y} gradient (phase encoding gradient) is turned on before signal readout, which causes phase encoding along the y axis, and allows sampling of signals located in different positions along the k_{y} axis. By repeating the series of G_{y}, Gₓ gradients and signal readout periods, a rectilinear, or Cartesian, trajectory is followed in k-space. After the k-space representation of the imaged object has been sufficiently sampled a 2D inverse Fourier transform provides the image of the object:

\hat{p}(r)=∫s(k)e^{i2πk·r}dk (6)

Discretizing this integral, we get:

$$\hat{p} ( {r}_{n} ) = {\sum }_{m = 1}^{M} s ( {k}_{m} ) {e}^{i2Π {k}_{m} \cdot {r}_{n}} \qquad ( 7 )$$

This equation is evaluated fast by a two-dimensional inverse fast Fourier transform (FFT).

In PROPELLER, Gₓ and G_{y} gradients with different amplitudes are combined in such a manner that the k-space samples are not located on a Cartesian grid (FIGS. 1, 2, 3). Thus, the final image cannot be reconstructed from the original PROPELLER k-space samples using a 2D inverse FFT as described above. Instead, conventional PROPELLER image reconstruction uses gridding, according to which k-space values on a Cartesian grid are first estimated from the PROPELLER samples, and then the 2D inverse FFT is applied on the k-space data residing on the Cartesian grid to reconstruct the final image. The gridding operation can be represented as:

M_{c}(kₓ,k_{y})={(Mₚ•W) [symbol] C}III [symbol] ¹C (8)

where, M_{c}(kₓ,k_{y}) is the k-space data on a Cartesian grid, Mₚ is the PROPELLER data, W is the weighting function that compensates for the non-uniform sampling density, C is the convolution function, III is the Cartesian grid, and [symbol] and [symbol] ¹ represent the convolution and deconvolution operation respectively.

The current invention differs from the above PROPELLER process by, upon acquiring a plurality of k-space data sets from the plurality of MR scans, transforming the plurality of k-space data sets to an image using an iterative reconstruction process. As described further below, in one embodiment of this invention, the iterative reconstruction process first constructs an image in image space using the acquired k-space data, and then modifies this image iteratively, in order to minimize the difference between the k-space representation of the image and the original measured k-space data, as well as the total energy over the image.

The goal in PROPELLER image reconstruction is to produce an image on a Cartesian grid from non-uniformly spaced k-space samples. If F(k) represents PROPELLER k-space samples (non-uniformly spaced), and f(x) is the reconstructed image (on a Cartesian grid), then:

F=φf (9)

where φ is the non-uniform Fourier transform and computes the transform of image f into non-uniformly spaced PROPELLER k-space samples. To estimate f from measured F, it is required to compute the inverse of φ, which is a computationally extensive operation. However, according to the method of this invention, f can be estimated iteratively by minimizing the difference between the k-space representation of the image and the original measured k-space data, as well as the total energy over the image, through minimization of the following cost function:

Θ(f)=½∥F_{measured}−φf∥w²+βR(f) (10)

This cost function consists of two terms. The first term is the weighted distance between the measured k-space data, F_{measured}, and the estimated k-space representation of the image produced in one iteration, F_{estimated}=φf. The second term is a quadratic penalty term, which represents the total energy over the image. β is the penalty value that controls the influence of the penalty term and balances the trade-off between the two terms. For higher β values, the cost function is significantly influenced by the penalty function, producing blurry images. In contrast, for lower penalty values, the contribution of the first term in the cost function is more significant, which may produce images with higher noise levels. A penalty value of 0.1 was found to provide a good balance between the two terms. The cost function in Equation (10) is desirably minimized using the conjugate gradient (CG) method with the known Fletcher-Reeves update formula.

In one embodiment of this invention, the non-uniform fast Fourier transform (NUFFT), Γ, is used instead of the non-uniform Fourier transform, φ, to rapidly and accurately evaluate the transformation off into non-uniformly spaced PROPELLER k-space samples. The NUFFT is achieved by projecting signal on over-sampled uniform Fourier basis γ, using standard FFT, followed by efficient interpolation:

F≅Γf=Iₙγf (11)

where Iₙ denotes the interpolation operator, which makes use of n neighboring k-space samples residing on an over-sampled Cartesian grid for approximation of the desired non-uniformly-spaced k-space samples. The interpolation coefficients Iₙ are computed using the min-max approach.

Once the percent difference of two images reconstructed in two consecutive iterations is lower than a pre-selected threshold, the reconstruction is complete. The resulting image is displayed as the magnetic resonance image.

The invention also contemplates software for use in implementing the above-described method. The software would be recorded on a recordable medium that can be executed on a data processor in combination with an MRI apparatus. As such, the software is loaded onto, for example, hard drives of data processors of existing MRI apparatuses to allow the image reconstruction method of this invention to be performed on existing machines without a change in hardware.

Thus, the invention provides a method for reducing PROPELLER MRI data acquisition times, by combining k-space under-sampling and iterative reconstruction using NUFFT, while maintaining similar image quality as in sufficient k-space sampling. PROPELLER imaging has a major advantage over conventional fast spin-echo (FSE) imaging in the fact that it is less sensitive to motion. This is a very crucial advantage, since oftentimes pediatric scans as well as scans on uncooperative subjects are severely compromised due to subject motion (even motion of few millimeters). With the method of this invention, PROPELLER imaging can be completed in equal or less time than FSE and has the imaging quality to replace FSE for clinical applications.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A method of obtaining a magnetic resonance (MR) image, the method comprising:
conducting a plurality of MR scans;
acquiring a plurality of k-space data sets from the plurality of MR scans;
transforming the plurality of k-space data sets to an image space using an iterative reconstruction process; and
displaying the magnetic resonance image.

2. The method of claim 1, wherein the plurality of MR scans comprises PROPELLER scans.

3. The method of claim 2, wherein the plurality of MR scans comprises a plurality of PROPELLER blades and the plurality of k-space data sets comprises a plurality of k-space lines.

4. The method of claim 3, wherein the plurality of k-space data sets comprises an under-sampled sampling scheme.

5. The method of claim 4, wherein the plurality of PROPELLER blade MR scans comprises less than 12 PROPELLER blade MR scans.

6. The method of claim 5, wherein each of the less than 12 PROPELLER blade MR scans comprises 16 lines per blade and 128 samples per line.

7. The method of claim 4, wherein the plurality of PROPELLER blades includes less than the number of blades necessary for sufficient k-space sampling.

8. The method of claim 4, wherein the plurality of k-space data sets has a sampling that satisfies Δk>1/FOV, where Δk is the maximum distance between adjacent samples in k-space and FOV is the field of view in the image space.

9. The method of claim 1, wherein the iterative reconstruction process comprises utilizing non-uniform fast Fourier transform.

10. The method of claim 1, wherein the iterative reconstruction process comprises minimizing a cost function.

11. The method of claim 10, wherein the iterative reconstruction process comprises minimizing a weighted sum of the total energy over the image and the difference between the k-space representation of the image in image space and the original measured k-space data or the total energy over the image.

12. The method of claim 10, wherein transforming the plurality of k-space data sets to the image space using the iterative reconstruction process comprises:
constructing an image in image space using the plurality of k-space data;
calculating a plurality of estimated k-space data sets from the image;
determining a difference between the plurality of k-space data sets and the estimated k-space data sets; and
minimizing the cost function by iterating the constructing, calculating and determining steps.

13. Software recorded on a computer readable medium and executable on a data processor for implementing the method of claim 1.

14. A method of obtaining a magnetic resonance (MR) image, the method comprising:
conducting a plurality of PROPELLER MR scans;
acquiring a plurality of k-space data sets from the plurality of MR scans;
transforming the plurality of k-space data sets to an image space by an iterative reconstruction process comprising non-uniform fast Fourier transform; and
displaying the magnetic resonance image.

15. The method of claim 14, wherein the plurality of k-space data sets comprises a plurality of k-space lines.

16. The method of claim 14, wherein the plurality of k-space data sets comprises an under-sampled sampling scheme.

17. The method of claim 14, wherein the plurality of PROPELLER blade MR scans comprises less than 12 PROPELLER blade MR scans, each including 16 lines per blade and 128 samples per line.

18. The method of claim 14, wherein the plurality of k-space data sets has a sampling that satisfies Δk>1/FOV, where Δk is the maximum distance between adjacent samples in k-space and FOV is the field of view in the image space.

19. The method of claim 14, further comprising minimizing a cost function.

20. The method of claim 14, further comprising minimizing a weighted sum of the total energy over the image and the difference between the k-space representation of the image in image space and the original measured k-space data or the total energy over the image.
